(19) United States
(12) Patent Application Publication
(10) Pub. No.: US 2024/0074805 A1
(43) Pub. Date: Mar. 7, 2024

(54) SYSTEMS AND METHODS FOR TREATING THE BLADDER WITH CONDENSABLE VAPOR

(71) Applicant: Boston Scientific Scimed, Inc., Maple Grove, MN (US)

(72) Inventors: Michael Hoey, Shoreview, MN (US); Maximillian D. FIORE, Eden Prairie, MN (US)

(73) Assignee: Boston Scientific Scimed, Inc., Maple Grove, MN (US)

(21) Appl. No.: 18/497,060

(22) Filed: Oct. 30, 2023

Related U.S. Application Data
(63) Continuation of application No. 17/647,219, filed on Jan. 6, 2022, now Pat. No. 11,864,810.
Application No. 17/647,219 is a continuation of application No. 16/891,262, filed on Jun. 3, 2020, now Pat. No. 11,246,641.
Application No. 16/891,262 is a continuation of application No. 15/154,536, filed on May 13, 2016, now Pat. No. 10,702,327.
(60) Provisional application No. 62/160,963, filed on May 13, 2015.

(51) Int. Cl. A61B 18/04

(52) U.S. Cl. CPC A61B 18/04; A61B 2018/00577

(57) ABSTRACT

A vapor delivery system and method is provided that is adapted for ablating bladder tissue to treat overactive bladder (OAB). The vapor delivery system includes an anchor tip configured anchor the system in the bladder while condensable vapor is delivered to target tissue. In one method, the vapor delivery system is advanced transurethrally into the patient to access the target tissue of the bladder, which can include a surface sensor of the bladder responsible for creating an urge incontinence sensation. The vapor delivery system includes a vapor source that provides a high quality vapor for delivery to tissue.

### CROSS REFERENCE TO RELATED APPLICATIONS

This application claims the benefit of U.S. Provisional Patent Application No. 62/160,963, filed May 13, 2015, titled “SYSTEMS AND METHODS FOR TREATING THE BLADDER WITH CONDENSABLE VAPOR”, which is incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present disclosure relates to devices and related methods for treatment of bladder using a minimally invasive approach. More specifically, this disclosure relates to treating overactive bladder with condensable vapor.

### BACKGROUND

Overactive Bladder (OAB) is a condition affecting millions of people. Symptoms include uncontrollable urges to urinate and incontinence. Frequent and sudden urges to urinate can negatively impact quality of life in those affected by the disorder.

Current treatment of OAB includes lifestyle changes including fluid restriction, avoidance of caffeine, and pelvic floor muscle exercise. Medications can also treat some symptoms of OAB but are only moderately effective. Surgical procedures have also been used including botox injections or electrical stimulation of the bladder, but the long-term effectiveness of these treatments is unknown.

It is postulated that OAB is caused by increased connectivity and excitability of both detrusor smooth muscle and nerves. Increased excitability and connectivity of nerves involved in micturition rely on growth factors that orchestrate neural plasticity. Neurotransmitters, prostaglandins, and growth factors, such as nerve growth factor, provide mechanisms for bidirectional communication between muscle or urothelium and nerve, leading to OAB with or without urge incontinence.

### SUMMARY OF THE DISCLOSURE

A method for treating overactive bladder is provided, comprising the steps of inserting a vapor delivery system into a urethra of a patient, advancing a distal portion of the vapor delivery system to a bladder of the patient, viewing the distal portion of the vapor delivery system with a camera disposed on or within the vapor delivery system, deploying a vapor delivery tip from the vapor delivery system into the bladder tissue to deform the tissue without penetrating the tissue, and delivering vapor into the bladder tissue to damage nerves, smooth muscle urothelium and other excitable tissue in the bladder.

In some embodiments, the delivering step comprises delivering between 1 calorie of energy and 500 calories of energy into the bladder at multiple locations with the idea of necrosing excitable tissues for the purpose of reducing the symptoms of overactive bladder, painful bladder syndrome and/or interstitial cystitis.

A method for treating overactive bladder in a patient is provided, comprising the steps of inserting a vapor delivery catheter into a urethra of the patient, advancing a distal anchor tip of the vapor delivery catheter transurethrally into a bladder of the patient, positioning the distal anchor tip on or adjacent to a target tissue including a surface sensor of the bladder responsible for creating an urge incontinence sensation, advancing the distal anchor tip into the target tissue to deform the bladder without puncturing the bladder, and delivering vapor through the vapor delivery system to the target tissue to ablate the target tissue including the surface sensor.

In some embodiments, the method further comprises delivering between 1 and 500 calories of energy to the target tissue.

In other embodiments, the method further comprises advancing the distal anchor tip less than 10 mm into the target tissue.

In alternative embodiments, the method further comprises advancing the distal anchor tip less than 5 mm into the target tissue.

In one embodiment, the distal anchor tip cannot extend more than 5 mm into the target tissue.

In another embodiment, the distal anchor tip cannot extend more than 1-2 mm into the target tissue.

In some embodiments, the advancing step further comprises advancing a needle of the distal anchor tip into the target tissue.

In another embodiment, the delivering step further comprises delivering vapor through vapor delivery ports disposed on a distal catheter tip of the vapor delivery system.

In other embodiments, the delivering step further comprises delivering vapor through at least one vapor delivery port disposed on the distal anchor tip of the vapor delivery system.

In one embodiment, the vapor is delivered through the vapor delivery ports to create one or more concentric spray patterns configured to form concentric treatment rings in the target tissue.

In some embodiments, the method further comprises ablating the target tissue to a depth of 1-3 mm.

A vapor delivery system is also provided, comprising a handle portion, an elongate flexible shaft connected to the handle portion, the elongate flexible shaft including a distal anchor tip configured to anchor the elongate flexible shaft into a bladder of a patient without puncturing through the bladder, a vapor source, at least one vapor delivery port disposed on the elongate flexible shaft and fluidly coupled to the vapor source, and an electronic controller operatively coupled to the vapor source and configured to deliver vapor through the at least one vapor delivery port to ablate the bladder.

In some embodiments, the distal anchor tip comprises a conical shape.

In other embodiments, the distal anchor tip comprises a pyramid shape.

In alternative embodiments, the distal anchor tip comprises a needle tip.

In one embodiment, the needle tip is configured to be retracted into the flexible elongate shaft during insertion into a patient and is configured to be advanced out of the flexible elongate shaft prior to vapor delivery.

The In some embodiments, the at least one vapor delivery port is arranged to create one or more concentric spray patterns configured to form concentric treatment rings in the bladder.

In other embodiments, the distal anchor tip is steerable.

In some embodiments, the distal anchor tip cannot extend more than 10 mm into the bladder.

In additional embodiments, the distal anchor tip cannot extend more than 1-5 mm into the bladder.

### BRIEF DESCRIPTION OF THE DRAWINGS

In order to better understand the invention and to see how it may be carried out in practice, some preferred embodiments are next described, by way of non-limiting examples only, with reference to the accompanying drawings, in which like reference characters denote corresponding features consistently throughout similar embodiments in the attached drawings.

FIG. 1 shows one embodiment of a vapor delivery system.

FIG. 2 shows a close-up view of a distal portion of the vapor delivery system.

FIG. 3 shows another embodiment of a distal portion of the vapor delivery system.

FIG. 4 shows yet another embodiment of a distal portion of the vapor delivery system.

FIGS. 5A-5B illustrate one method of treatment using the vapor delivery system.

FIG. 6 is a flowchart illustrating a method of treatment using the vapor delivery system.

### DETAILED DESCRIPTION

In general, one method for treating overactive bladder comprises introducing a heated vapor into contact with excitable tissue near the surface of the urothelium in the bladder that create the urge incontinence sensation. The method can cause localized ablation of bladder tissue to minimize urge incontinence or overactive bladder (OAB), painful bladder syndrome (PBS) and/or interstitial cystitis. The present disclosure is directed to the treatment of OAB, and more particularly for transurethrally ablating excitable tissue structures in the bladder that are responsible for urge incontinence.

The system can include a vapor delivery mechanism that delivers vapor media, including water vapor. The system can utilize a vapor source configured to provide vapor having a temperature of at least 60-140° C. In another embodiment, the system further comprises a computer controller configured to deliver vapor for an interval ranging from 1 second to 30 seconds.

In some embodiments, the system further comprises a source of a pharmacologic agent or other chemical agent or compound for delivery with the vapor. These agents include, without limitation, an anesthetic, an antibiotic or a toxin such as Botox®, or a chemical agent that can treat cancerous tissue cells. The agent also can be a sealant, an adhesive, a glue, a superglue, an anti-inflammatory, an antibiotic, or the like.

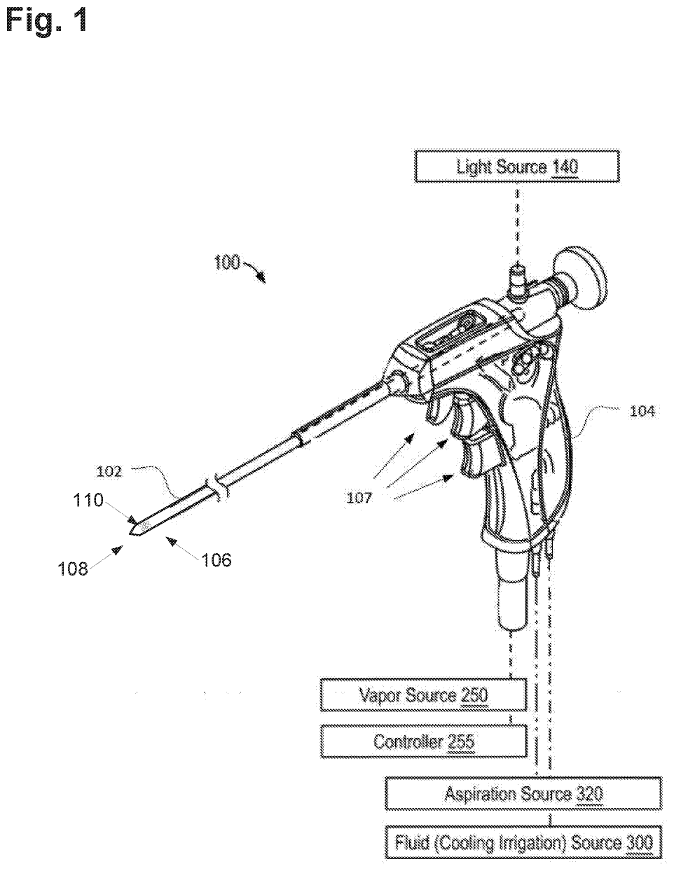

FIG. 1 shows one embodiment of a vapor delivery system. Vapor delivery system 100 can have an elongate flexible shaft 102 configured for insertion into the urethra of a patient and a handle portion 104 for gripping with a human hand. The vapor system 100 can include a distal catheter tip 106 that includes an anchor tip 108 and one or more vapor delivery ports 110. In some embodiments, the distal catheter tip can be deformable and/or steerable.

The one or more vapor delivery ports 110 are fluidly coupled to the vapor source and can be configured to deliver a flow of vapor media from a vapor source 250 through the vapor elongate flexible shaft 102 into bladder tissue. The vapor delivery system can further include an aspiration source 320 configured to aspirate tissue and/or fluids from the patient (e.g., either through the vapor delivery ports or through separate aspiration ports in the distal catheter tip), and can also include a fluid source 300 configured to deliver a fluid or cooling irrigation to the tissue before, during, and/or after vapor delivery.

The vapor delivery system 100 of FIG. 1 can include a plurality of actuation devices 107, such as triggers, buttons, or levers, which can be configured to actuate the various functions of the system. For example, the actuation devices can be configured to steer the distal catheter tip, and start/stop the flow of vapor, aspiration, and/or the cooling irrigation.

Vapor source 250 is provided for generating and/or delivering a vapor media through the distal catheter tip to ablate or damage tissue. The vapor source can be a vapor generator that can deliver a vapor media, such as water vapor, that has a precisely controlled quality to provide a precise amount of thermal energy delivery, for example measured in calories per second. In some embodiments, the vapor source can comprise an inductive heating system in which a flow media is inductively heated to generate a condensable vapor. The vapor source can be external to the vapor delivery system, or alternatively, can be integrated into the handle and or elongate flexible shaft of the vapor delivery system.

The controller 255 can be set to control the various parameters of vapor delivery, for example, the controller can be set to delivery vapor media for a selected treatment interval, a selected pressure, or selected vapor quality. Further details on the vapor delivery system, the vapor generator, and how vapor and fluid are delivered to tissue can be found in U.S. Pat. No. 8,273,079 and PCT Publication No. WO 2013/040209, both of which are incorporated by reference. In some embodiments, the electronic controller can also control the aspiration and/or cooling irrigation functions of the vapor delivery system.

As described above, the vapor delivery system can be connected to a vapor source 250, an aspiration source 320, a fluid or irrigation source 300, a light source 140, and an electronic controller 255 configured to control generation and delivery of vapor from the vapor source, through a lumen of the shaft, through the distal catheter tip, and into tissue. In some embodiments, the electronic controller can be disposed on or in the vapor delivery system, and in other embodiments the electronic controller can be disposed separate from the system.

Referring still to FIG. 1, the fluid or irrigation source 300 can provide a fluid, such as saline, through a separate lumen in the shaft to provide irrigation and flushing to tissue during insertion of the system and during vapor delivery to tissue. In some embodiments, the irrigation can be used to clear blood and debris from tissue lumens to increase visibility. The irrigation can also provide cooling to the urethra and/or bladder of the patient, both via direct contact of the irrigation fluid with the tissue as well as cooling the shaft of the vapor delivery system as the fluid flows from the irrigation source through the shaft and into contact with the tissue.

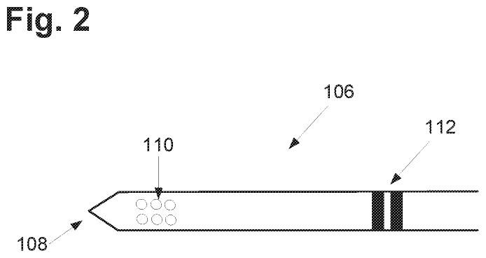

FIG. 2 shows a close-up view of the distal catheter tip 106 of the vapor delivery system. The elongate shaft and the distal catheter tip are sized and configured to be inserted into a lumen of a flexible cystoscope. The distal catheter tip can further include a distal anchor tip 108 that is configured to be forced or anchored partially into tissue of the bladder to deform the tissue without penetrating the tissue. When the distal anchor tip 108 is deforms the bladder it can anchor the vapor delivery system in the target tissue of the bladder. In one embodiment, the pointed tip can be a conical or pyramid shaped pointed tip. In embodiments where the pointed tip is a pyramid shaped pointed tip, the pyramid shape can include two, three, or more faces. By deforming the tissue without penetrating the tissue, the pointed anchor tip can be anchored in the bladder tissue near the target nerves or sensors to be treated without compromising the structural integrity of the bladder.

When the pointed anchor tip is anchored in the bladder tissue, vapor can be delivered from the vapor source to the bladder tissue through the one or more vapor delivery ports 110. In some embodiments, the vapor ports can be disposed proximally to the anchor tip 108 on the shaft. In other embodiments, the vapor ports can also be disposed on the anchor tip itself.

The position of the vapor ports with respect to the anchor tip determines the vapor spray pattern, which can be adjusted and designed according to the desired thermal ablation in the tissue. In one embodiment, the vapor ports can be arranged in a concentric manner on the tip and/or shaft to create one or more concentric spray patterns configured to form concentric treatment rings in the bladder tissue. Vapor can be delivered to the bladder to ablate the tissue to a specific depth that damages the targeted bladder sensors without ablating the full thickness of the bladder tissue. For example, in some embodiments, the vapor can be delivered to the bladder tissue to ablate a depth of 1-6 mm of bladder tissue. It is desired to treat only a small depth of tissue to achieve the goal of damaging the sensors without ablating a whole through the entirety of the bladder tissue.

The distal catheter tip can optionally include visualization features 112, which can be markings on the catheter tip. The visualization features 112 can include, for example, stripes, shapes, colors, or other visual features disposed directly on the distal catheter tip. When the distal catheter tip is extended beyond the cystoscope during an ablation procedure, the visualization features can be viewed under visualization (e.g., with a camera) of the cystoscope during to determine when the distal catheter tip is advanced the proper distance beyond the cystoscope. Also, the markings on the catheter can be used to guide the clinician in placing the catheter in the proper orientation to deliver a vapor pattern in a prescribed pattern. For example, the visualization features can include a feature to indicate where the vapor delivery ports are oriented on the distal catheter tip, or the visualization features may be disposed on the shaft only in sections where vapor delivery ports are located.

The pointed anchor tip of the vapor delivery system prevents slippage and anchors the vapor delivery system when a clinician pushes the vapor delivery system into the bladder. As the clinician pushes harder, the vapor delivery system deforms the bladder as it continues to extend beyond the cystoscope. The clinician can determine the level of bladder deformation by observing the distance of the visualization features from the cystoscope. The amount of bladder deformation also determines the pattern of vapor deposition from the vapor delivery ports onto the bladder tissue. Vapor can be delivered from the vapor delivery system onto or into the bladder tissue when the appropriate pattern of vapor deposition is achieved.

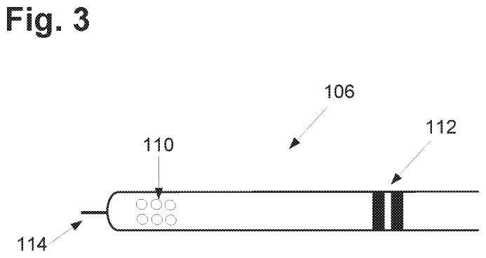

FIG. 3 shows an alternate embodiment of a distal catheter tip 106, which includes a needle anchor tip 114 configured to be forced into the tissue of the bladder to deform the tissue without fully penetrating the tissue. The needle anchor tip 114 can operate in a similar manner to the pointed anchor tip described above. In some embodiments, the needle anchor tip 114 can include one or more vapor delivery ports disposed on or in the needle, or at the very distal tip of the needle.

In some embodiments, the needle anchor tip can be retracted into the distal catheter tip during navigation to the bladder, and advanced out of the catheter tip to anchor the system for vapor delivery. For example, an actuation device on the vapor delivery system can control advancement and retraction of the needle. In some embodiments, the needle can be designed to advance a limited distance from the distal tip of the catheter so as to not fully penetrate through or puncture bladder tissue. For example, bladder tissue can typically range from 5-15 mm in thickness. In some embodiments, the needle anchor tip 114 cannot extend more than 10 mm into the tissue so as to not puncture through the bladder tissue. In some embodiments, the clinician can determine the thickness of the patient's bladder (such as with visualization) and adjust the length of the needle anchor tip to be less than the thickness of the bladder. In other embodiments, the needle anchor tip cannot extend more than 1-5 mm into the tissue, which can be sufficient to anchor the catheter tip in the bladder tissue but not be long enough to puncture the bladder tissue in the vast majority of potential patients. Once the system is anchored in the tissue with the needle anchor tip, vapor can be delivered to the bladder tissue through vapor delivery ports 110.

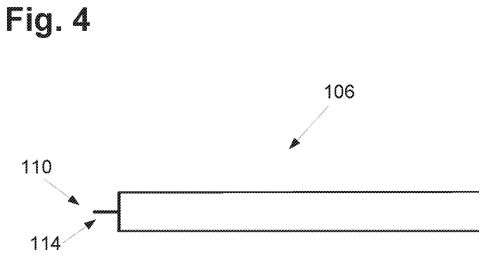

FIG. 4 shows another embodiment of a distal catheter tip 106 having a needle anchor tip 114, similar to the embodiment described above in FIG. 3. However in this embodiment, the vapor delivery port(s) 110 are located on or in the needle anchor tip 114 itself, instead of being located on the catheter shaft. The vapor delivery port(s) 110 can be located at the distal end of the needle anchor tip 114, for example. This configuration allows for the needle anchor tip 114 to anchor the distal catheter tip in tissue, and to deliver vapor through the vapor delivery ports 110 directly into the bladder tissue.

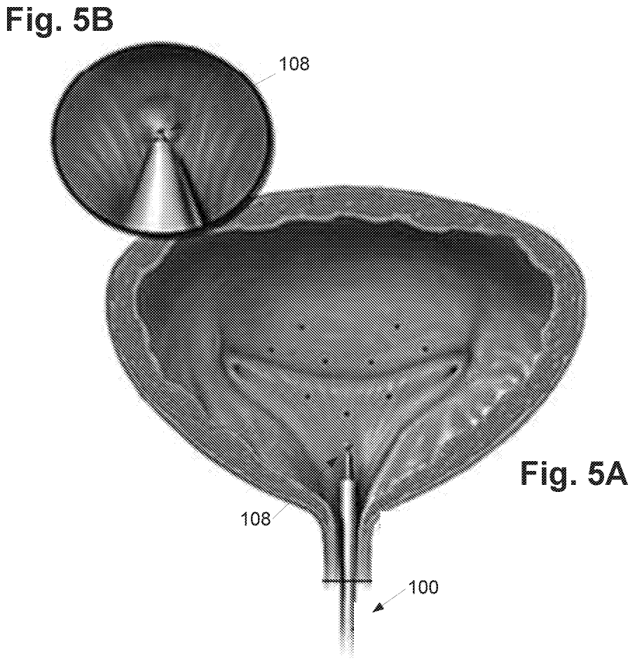

Referring to FIG. 5, the device and method of this disclosure provide a precise, controlled thermal ablative treatment of tissue in the bladder for the treatment of OAB. In particular, the ablative treatment is configured to ablate surface sensors in or near the bladder that create the urge incontinence sensation to treat OAB.

Sensory perception of the urge to urinate may be mediated by two bladder sensors. The first sensor, located at the trigone and posterior urethra, is sensitive to small changes in pressure and may function as an early warning system of bladder filling. Some instances of urge incontinence result when the early warning system fails and detrusor contraction occurs just shortly after the second sensor is stimulated. Thus, two forms of urge incontinence may be associated with loss of the first sensor: one with and the other without associated frequency, with the difference being the presence or absence of detrusor instability. The method described herein describes a technique for ablating sensors with vapor to achieve a permanent effect to minimize urge incontinence or OAB.

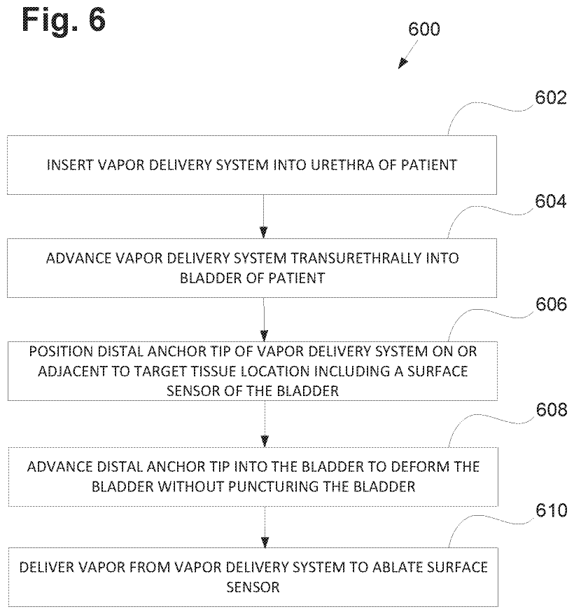

In FIGS. 5A-5B shows a vapor delivery system 100 and anchor tip 108 inserted into a bladder of a patient. FIG. 5B shows a close-up view of the anchor tip. FIG. 6 shows a flowchart 600 describing a method for treating overactive bladder in a patient, which can comprise the following steps: First, (at step 602 of flowchart 600 in FIG. 6) a shaft including an anchor tip 108 of a vapor delivery system 100, such as the one described above, can be inserted into a urethra of the patient. In some embodiments, the shaft of the vapor delivery system is inserted into a cystoscope which is inserted into the urethra of the patient. In other embodiments, the shaft of the vapor delivery system itself can be inserted into the urethra.

Next, (at step 604 of flowchart 600 in FIG. 6) the vapor delivery system and/or cystoscope can be advanced transurethrally into the patient until a distal portion of the vapor delivery system including the anchor tip and/or cystoscope is positioned within the bladder.

Next, (at step 606 of flowchart 600 in FIG. 6) a distal anchor tip of the vapor delivery system can be advanced and positioned on or adjacent to a target location including a surface sensor of the bladder responsible for creating an urge incontinence sensation. In embodiments when a cystoscope is used, the distal anchor tip of the vapor delivery system can be extended beyond the end of the cystoscope under visualization through the cystoscope.

Next, (at step 608 of flowchart 600 in FIG. 6) the distal anchor tip can be advanced into the bladder tissue on or adjacent to the surface sensor to deform the bladder tissue without puncturing the bladder tissue. In some embodiments, the distal anchor tip is steerable, and can be turned or deformed to accurately anchor the tip on, near, or adjacent to the surface sensor to be treated.

Finally, (at step 610 of flowchart 600 in FIG. 6) vapor can be delivered through the vapor delivery system to the target tissue including a surface sensor to ablate the surface sensor. Sufficient energy can be delivered by the vapor to the surface sensor to ablate the surface sensor without causing injury to the bladder. In some embodiments, the vapor delivery system is configured to deliver between 1-500 calories of energy to the target tissue. This process can be repeated with other surface sensors in the bladder until all the target surface sensors have been treated.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

1.-20. (canceled)

21. A vapor delivery device, comprising:
a shaft defining a plurality of vapor delivery ports; and
a distal tip, wherein the distal tip is narrower than a portion of the shaft proximal of the distal tip, wherein the portion of the shaft proximal of the distal tip defines the plurality of vapor delivery ports, and wherein the distal tip is distal to each of the plurality of vapor delivery ports.

22. The vapor delivery device of claim 21, wherein the distal tip tapers to a pointed distal end.

23. The vapor delivery device of claim 22, wherein the distal tip has a conical shape.

24. The vapor delivery device of claim 22, wherein the distal tip has a pyramid shape.

25. The vapor delivery device of claim 22, wherein the pointed distal end is a distalmost end of the vapor delivery device.

26. The vapor delivery device of claim 21, wherein a visual feature is disposed on the shaft, wherein the visual feature is configured to be visualized by a camera to determine positioning of the shaft.

27. The vapor delivery device of claim 21, wherein the distal tip is a needle, and wherein the needle is configured to be advanced and retracted relative to the shaft.

28. The vapor delivery device of claim 27, wherein the shaft or the needle is configured so as to inhibit the needle from advancing more than 15 mm distally past a distal end of the shaft.

29. The vapor delivery device of claim 21, further comprising a vapor source in fluid communication with the plurality of vapor delivery ports.

30. The vapor delivery device of claim 21, wherein the distal tip is configured to deform a tissue of a bladder without puncturing through a thickness of the tissue.

31. The vapor delivery device of claim 21, wherein each of the distal tip and the shaft is a portion of a catheter.

32. A vapor delivery device comprising:
a shaft defining a plurality of vapor delivery ports; and
a needle tip, wherein the needle tip is configured to be advanced and retracted relative to the shaft.

33. The vapor delivery device of claim 32, wherein, upon advancement of the needle tip, an exposed portion of the needle tip is distal to a distalmost end of the shaft.

34. The vapor delivery device of claim 33, wherein the exposed portion is distal to each of the plurality of vapor delivery ports.

35. The vapor delivery device of claim 33, wherein the shaft or the needle tip is configured so as to inhibit the exposed portion from exceeding a length of 15 mm.

36. the vapor delivery device of claim 32, further comprising a vapor source in fluid communication with the plurality of vapor delivery ports.

37. The vapor delivery device of claim 32, wherein the needle tip is configured to deform a tissue of a bladder without puncturing through a thickness of the tissue.

38. A vapor delivery device comprising:
a vapor source;
a shaft defining a plurality of vapor delivery ports, wherein the plurality of vapor delivery ports are in fluid communication with the vapor source; and
a pointed distal tip, wherein the pointed distal tip is distal to each of the plurality of vapor delivery ports.

39. The vapor delivery device of claim 38, wherein the pointed distal tip is a distalmost end of the vapor delivery device.

40. The vapor delivery device of claim 38, wherein each of the pointed distal tip and the shaft is a portion of a catheter.
